(19) United States
(12) Patent Application Publication
(10) Pub. No.: US 2006/0153890 A1
(43) Pub. Date: Jul. 13, 2006

(54) Insect repellent

(76) Inventor: Moriyuki Yamane, Hikawa-gun (JP)

Correspondence Address: ARMSTRONG, KRATZ, QUINTOS, HANSON & BROOKS, LLP, 1725 K STREET, NW, SUITE 1000, WASHINGTON, DC, 20006, US

(21) Appl. No.: 11/032,223

(22) Filed: Jan. 11, 2005

(51) Int. Cl. A61K 36/00; A01N 65/00

(52) U.S. Cl. 424/405; 424/725

(57) ABSTRACT

The present invention provides an insect repellent derivative from natural plants and having novel and excellent effects.

An extract liquid obtained by applying an ordinary extracting process to leaves, flowers, branches, stems, or roots of the spiraea group plants by use of water, or an organic solvent such as a low alcohol, or a water-containing low alcohol;
an extract liquid obtained by boiling or steaming leaves, flowers, branches, stems, or roots of the spiraea group plants;
or a supernatant or a squeezed liquid obtained by squeezing leaves, flowers, branches, stems, or roots of the spiraea group plants or grinding together with water and other solvent leaves, flowers, branches, stems, or roots of the spiraea group plants are used as an insect repellent as it is or in the form of being condensed or powdered.

### BACKGROUND OF THE INVENTION

1. Field of the Invention

The present invention relates to a novel insect repellent derived from plants, in particular, to an insect repellent containing as an effective content an extract of leaves, flowers, branches, stems (stalks), or roots of the Spiraea group plants.

2. Prior Art

Hitherto widely used as an insecticide or an insect repellent are naphthalene, “paradichlorbenzol”, etc. Insect repellents in the form of a spray, gel, or foam are extensively applied to outdoor use. These insect repellents are provided industrially, cheaply and at a large amount. Also long known to public are insecticides and insect repellents derived from natural plants, such as camphor, a laurel, eucalyptus oil and various herbs. “Paradichlorbenzol” has been recently pointed as problematic in being cancer-causing or carcinogenic. “DEET” (N,N-diethyl-m toruamido) has been found as causing various troubles such as high mutation, headache, eczema, a defect of memory, etc, which causes the insect repellents derived from natural materials such as plant to become popular or given attention.

An insect repellent which uses or comprises: solely eucalyptus oil; a mixture of oils of eucalyptus, lemon grass, mint, and cloves; a conifer sap; or herbs is commercially available recently. The insect repellent products using these oils have effect of repellency that does not continue so long (i.e., kept about one to two hours after sprayed) and are expensive, and also substantially poor in the effect of repellency in comparison with the foregoing goods employing chemicals.

From the above standpoint, the inventor attempted various tests using extracts derived from various plants close to us and found the fact that spiraea plant extracts shows excellent repellence of harmful insects, and accordingly achieved the present invention.

First, for the purpose of repelling cabbage butterflies to prevent them from blowing or lay eggs on cabbage, the inventor scattered on the cabbage field cut leaves of various plants growing on gardens and fields subjected to less damages from harmful insects, such as a low striped bamboo (Sasa albo-marginata Makino et Shibata.), Pittosporum Tobira Ait., a laurel, Spiraea Thunbergii Sieb., or the like. Some sorts of those plants showed a somewhat effect. In order to obtain an essence of these plants, their leaves were shattered in existence of water, and an extract liquid was put and left in a bottle. Most of those plants had mold or became rotten and smelled bad. Only the Spiraea Thunbergii Sieb., among the plants did not show such changes.

In addition, there was found such fact that the spiraea extract liquid was not likely to evaporate even when left in a beaker under a burning sun. Then, a proper amount of the extract liquid was applied on an iron plate and left similarly. The extract liquid increased the character of being viscous while water content evaporates, and finally looked like starch syrup after about one or two days. The starch syrup-like substance was dissolved quickly in water. Also, this substance showed deliquescence and adsorbed water content from the atmosphere having high humidity in a rainy day to be softened.

Moreover, there were found such facts that leaf beetles and flies do not come near the beaker in which the extract liquid is placed, or boiled rice or confectionery sprinkled with the extract liquid and that ants disappear before we knew it from around a place where the extract liquid is spilt. This means that the substance contained in live leaves of the Spiraea Thunbergii Sieb. has a function of restraining water from evaporating and also another function of repelling insects or the like. Other plants, such as Spiraea cantoniensis Lour., Spiraea japonica L. fil, and Spiraea prunifolia Sieb. et Zucc., belonging to the spiraea group plants in addition to the Spiraea Thunbergii Sieb. had and showed the same functions. At present the substance has not yet been identified. It is not clear with which way the foregoing facts or effects can be provided, namely, by a single substance or a multiplier effect of a plurality of substances.

Spiraea Thunbergii Sieb., Spiraea cantoniensis Lour., Spiraea japonica L. fil, and Spiraea prunifolia Sieb. et Zucc., are widely and generally distributed or grow in Japan as wild plants or garden trees. Flowers themselves of these plants as garden trees or in flower arrangement are loved by peoples. But, those plants are not usable in other ways and not at all described in any encyclopedias or dictionaries of medicinal herbs. And as far as the inventor knows, such fact has hitherto not at all been known that extracts from those plants have an effect of repelling insects. Spiraea Thunbergii Sieb. was thereafter found to have an antibacterial effect or action.

The reason why the foregoing extract liquid does not rot is inferred to be thanks to the antibacterial effect. The antibacterial components can be obtained by an extracting process from fresh leaves, trunks, fruits or flowers of the spiraea group plants by use of ethanol or butanol, followed by an eluting process using ethylacetate-benzene. But, the antibacterial components are not contained in an extract obtained through an extracting process using water. However, that extract through the extracting process using water has similarly the bactericidal or germicidal effect or action so that the extract is inferred to contain an antibacterial component different from those known to public.

### SUMMARY OF THE INVENTION

The present invention provides an insect repellent derived from plants and superior in the insect repelling effect and in keeping of the effect, more particularly, an insect repellent employing as an effective component an extract from leaves, flowers, branches and stems and roots of the spiraea group plants or a treated product of the extract.

### BRIEF DESCRIPTION OF THE DRAWINGS

FIG. 1 is a plan view showing an experiment using the insect repellent according to the present invention.

FIG. 2 is a plan view showing another experiment using the insect repellent according to the present invention.

### DETAILED DESCRIPTION OF THE INVENTION

Next, the present invention will be detailed with referring to the case of Spiraea Thunbergii Sieb. Spiraea Thunbergii Sieb. which is also called Spiraea prunifolia Sieb. et Zucc. blossoms to have grouped white flowers together with young leaves in March to April. The stems extend plurally from roots vertically in 1 to 3 m height and leaves gradually become into deep green and begin to yellow in the autumn and to fall then.

Initially, the inventor collected live leaves and flowers of Spiraea Thunbergii Sieb. to grind them together with water using a mixer and used the supernatant liquid in the experiments. There were found that a meterial obtained the live leaves and flowers merely soaked in water several days showed the similar insect repellency effect which is though in fact relatively poor, and another material obtained from the live leaves and flowers boiled with water and a further material from the foregoing supernatant heated to be condensed showed the insect repellency effect to some extent. The liquid derived from the grinding (the mixer process) of live leaves and flowers of Spiraea Thunbergii Sieb. together with water separates into the supernatant and fibrous material (the latter sinks) within one to several days. The supernatant becomes transparent and dark brown (like the color of whiskey) in about one month.

The supernatant is first thin brown and has the raw smell of the plant, and then becomes transparent dark brown as matured (after about one month) having the smell of a perilla and a special flavor mixing the perilla and plum liqueur. The supernatant when kept in this state even with three years passing does not rot. And the insect repellency effect becomes higher gradually after the time about one month passing. Such changes are inferred to be from a chemical change or a behavior of enzyme. Taste of the supernatant does also change as somewhat astringent and bitter. The supernatant as matured is 4.0 to 5.0 pH and stable at around 4.5 pH.

The supernatant when water content evaporates has a small amount of viscous solution on the bottom of the container. The solution smells similarly to the supernatant and forms again the original supernatant when water added. The solution and also the re-formed supernatant show similarly the insect repellency effect. From this fact, it is appreciated that the extracts from plants according to the present invention include a humidity keeping effect or an effect of wrapping-in chemical substances.

Residues (more than one month passed after the supernatant provided) with the supernatant being removed is regarded to have the similar insect repellency effect to the supernatant. The residue placed on a dish and left in a room shows the insect repellency effect. The residue may be used in the form of sticks of incense provided by fully drying the residue.

In case that Spiraea Thunbergii Sieb. live leaves and flowers are subjected to the mixer grinding process using ethanol as solvent in stead of water, fractions of the supernatant are higher in evaporation speed than the foregoing extracting case using water, resulting in leaving a stiff and dry residue. The solvent may further employ lower alcohol such as methanol, propyl alcohol, or isopropyl alcohol, or water containing lower alcohol. Also may employ are various organic solvent such as acetone, methyl ethyl ketone (MEK), or ether, or multivalent alcohol such as glycerin or the like. For an insect repelling agent in the type to be applied to human skin or for those to be sprayed in kitchens where food and drink are placed, the extracting process may be preferably performed, in consideration of safety, by using water, water-containing methanol or methanol as the solvent. To be noted is that in case of using alcohol or organic solvents, chlorophyll may be much dissolved to cause the extract liquid to be in vivid green. Meanwhile, the extract liquid derived from the mixer grinding process using water is initially slightly thin green and then becomes green brown.

Parts of Spiraea Thunbergii Sieb. usable in the extracting process may be its leaves, flowers, branches, stems, or roots. Leaves, particularly, fresh live leaves are most preferable for providing the insect repellency effect.

The supernatant (the extract liquid) as matured does not at all have rotting or mold even when kept as it is at a normal room temperature with its container having no lid. Moreover, as foregoing, the extract liquid has a faint smell of plants such as plum fruits, Perilla leaves, etc. The smell of the extract liquid when users are sick of it can be removed by that the material plants together with a small amount of laurel leaves, peppermint or other herbs are subjected to the foregoing mixer grinding process, or by that a trace amount of an extract or essence from plants having fragrance such as herbs is added.

The “treated product” of the extract may be those obtained by condensing, diluting, refining or powdering the extract liquid. The condensing does mean to heat the extract liquid so as to reduce water content. The condensing may preferably be performed under reduced pressure to avoid influence of heat of components. The diluting may be carried out with water or alcohol and in a free proportion and the extract liquid when diluted has a lowered insect repellency effect. The powdering may employ vacuum drying of the extract liquid since an ordinary hot-air drying is hard to be taken. These methods of treatment may be selectively chosen according to specific application or usages of the extract.

The extract liquid according to the present invention has the insect repellency effect but not an insecticidal effect. In this sense, the extract liquid according to the present invention is different from the insecticide such as naphthalene and camphor. Thus, even when the extract liquid is sprayed over ants marching in the garden or in the kitchen, the ants do neither die nor disrupt the line or become unruly. The ants go through the sprayed extract liquid in a manner of having no influence. However, after a short time, ants disappear from around there without our recognizing it. Moreover, mosquitoes and flies do not die even when they are directly sprayed with the extract liquid. But, mosquitoes and flies do not at all approach a place where there is suspended a piece of cloth over which the extract liquid is sprayed or which cloth is soaked in the extract liquid. In addition, small animals, generally called bugs or insects such as spiders, Armadillidium, Meghimatium, (these insects, bugs and small animals are also called “hygienically” harmful insects or “uncomfortable” harmful insects) other than the ordinary insects will disappear, before we know it, from the place where the extract liquid is sprayed or applied. Reasons why these phenomena arise have not been defined and it is so inferred that the extract liquid emits an odor that those small animals dislike. A component of the odor can be sensed by human being and is similar to a smell of plum fruits and plum liqueur. It has not been clarified in which way the odor that the small animals dislike as above is provided by a sole substance, a plurality of substances, or their interaction. But, not all the insects or the like are subjected to the insect repellency effect of the extract liquid according to the present invention. Crickets and dragonflies do not escape from but appear to come near the extract liquid.

The extract liquid according to the present invention does not have the insecticidal effect but has the bactericidal effect and the antibacterial effect as foregoing. Hence, even when raw waste is kept in the kitchen for a few days, the smell from rotting does not arise and flies and small flies are prevented from coming near and flyblowing, thereby enabling the circumstance to be kept quite hygienically. The extract liquid or the treated product thereof may be used being mixed with an insecticidal component. In this case, the mixture has also the insecticidal effect.

In detail, the extract liquid according to the present invention may be provided with an insecticidal efficiency by adding chemical synthetic products such as “pyrethroid”, organic phosphorus or organic chlorine insecticides, or plant essences having insecticidal efficiency such as an insect powder plant, or NEEM (Japanese Patent Publication No. Hei 7-39332 official gazette), or Ardisia crispa A. DC. (Japanese Unexamined Patent Application No. Sho 59-128319), or by performing the extracting process from the material plants mixing with leaves and branches of plants having insecticidal efficiency such as the insect powder plant, NEEM or Ardisia crispa A. DC. From the same standpoint, the extracting process may be performed with addition of chemical insect repellency agent such as DEET or the like or plants essences having insects repelling efficiency such as a laurel, Juniperus chinensis L., Pittosporum Tobira Ait., a gingko tree, or a mugwort, and mixing with leaves or the like of those plants, in order to improve the insect repellency effect. Furthermore, these effects may be reinforced by adding an antibacterial substance or a humidity keeping substance. Besides, in case that the extracting process is performed using also leaves or the like of other plants, when the proportion of such additional plants is higher, the extract liquid tends to rot. Mixing ratio of such additional plants is preferably 40% or less, particularly, 30% or less. To be noted is that it is preferable to refrain from adding any chemical products and synthetic products if possible.

The extract liquid is quite high in an efficiency of permeating wood and bamboo. Also, the extract liquid is not likely to evaporate, does not completely dry up, and has a bactericidal effect and an antibacterial effect. Thus, building materials when applied with the extract liquid according to the present invention have no fear of being affected or invaded with white ants and rot-causing bacteria. The insect repellency effect is kept substantially semi-permanently unless the applied extract liquid is washed out with water.

### EFFECT OF THE INVENTION

As explained above, the present invention relates to an insect repelling agent using as an effective component the extract from leaves, flowers, branches, stems, or roots of the spiraea group plants.

Hence, the present invention has the following effects.
(1) The insect repellent according to the present invention has an absolute repellency effect against mosquitoes, flies, ants, small flies, Armadillidium, Meghimatium, snails, and butterflies, particularly, against mosquitoes.
(2) The invention is very high in safety since it can be obtained from natural plants but not chemical products such as naphthalene, paradichlorbenzol or DEET. The invention does not include toxicity (insecticidal components) in comparison with the similar plants-derivative insect powder plant or camphor and is safe even when put on skin of human being or food.
(3) The insect repellency effect of the invention is quite high and not lower than the insect repelling sprays hitherto provided having DEET as the effective component. The insect repellency effect of the invention is further kept long in comparison with the hitherto provided insect repelling sprays unless the invention is washed out with water.
(4) The extract according to the present invention which itself has a bactericidal efficiency and an antibacterial efficiency does not need a preservation agent and also is not at all problematical even when people smells at the extract for many hours since the extract has no toxicity. Furthermore, the extract is safe even when sprayed for example in a kitchen, namely, near a place food is placed.
(5) The spiraea group plants such as Spiraea Thunbergii Sieb., Spiraea cantoniensis Lour., Spiraea japonica L. fil., and Spiraea prunifolia Sieb. et Zucc., are broadly seen as wild or garden trees and suitable for cultivation in a fallow field or fields located among the mountains. The plants grow very quickly, give no trouble and are available in a large amount at a low cost.
(6) The extract according to the present invention can be readily obtained with a simple operation and device. Thus, a cost to process is low or cheap.
(7) In case of using water as solvent, the cost is quite cheap and the extracting process can be carried out easily and in safety. And a secondary reaction is restrained by use of solvent, thereby the extract superior in safety can be obtained.
(8) Even in case of using water as solvent, the extract liquid does not rot and not change of color for several years (it has been confirmed as three years) and the insect repellency effect can be kept.
(9) The extract liquid of the invention is capable of being condensed, so that it is advantageously transported at a low cost to foreign countries where people are troubled with insects such as mosquitoes, flies and the like.

In addition, the insect repellent according to the present invention has the particular effect to the harmful insects, particularly, to mosquitoes. And since the invention is derived from plants, the invention is quite higher in the safety in comparison with the hitherto provided DEET or the like. Besides, the spiraea group plants such as Spiraea Thunbergii Sieb. and the like which are the material for the insect repellent according to the present invention are widely seen as wild or garden trees, are suitable for cultivation in fallow fields and fields among the mountains, do grow quickly, give no trouble, and are available in a large amount at a low cost, and also are readily provided with a simple operation and device at a cheaper cost to process.

### PREFERRED EMBODIMENTS OF THE INVENTION

Fresh live leaves of Spiraea Thunbergii Sieb. are sufficiently washed with water and then filled together with water into a mixer to be ground and agitated for three to five minutes. The liquid is placed in a container and left at rest for one to several days to cause separation into solid and liquid phases. Agitation is carried out when fibers float up. A transparent dark brown liquid was obtained in about one month. The transparent dark brown liquid shows an excellent repelling effect to harmful insects, particularly, to mosquitoes. After one to two months, the liquid became a very much transparent solution having a particular smell similar to plum liqueur and a reddish brown color and showing an excellent insect-repelling effect.

### EXAMPLE 1

### Manufacturing Method 1

Next, the present invention will be further detailed with referring to the specific examples. Fresh live leaves 50 g of Spiraea Thunbergii Sieb. are sufficiently washed with water and then filled into a mixer, with water 300 g poured, to be ground and agitated for three to five minutes. The liquid is placed in a container and left at rest for one to several days to cause separation into solid and liquid phases. Agitation is carried out when fibers float up. This product smells a raw smell of live leaves and becomes a transparent solution having reddish brown color like whiskies in about one month. This solution smells like plum fruits. The supernatant 300 cc which merely left at rest for several days was placed in a bottle and left at rest in the sun from May to June, resulting in that the supernatant reduced in quantity to about 270 cc in one month.

In grinding with the mixer, water is used in the extent 200 to 1000 cc with respect to live leaves 50 g. Squeezed solution provides highly condensed liquid.

### EXAMPLE 2

### Manufacturing Method 2

Flowers of Spiraea Thunbergii Sieb. and leaves of Spiraea cantoniensis Lour. are separately processed in the similar way to Example 1 to obtain transparent solutions. A solution provided from the former material has a thinner smell, after one month, in comparison with Example 1 and the insect repelling effect being poor. Another solution provided from the latter material smells the similar smell, after one month, but having a slightly different metal-like smell. The insect repelling effect was the same as or higher than Example 1. In case of using branches, residue is generated much and mold is likely to gather.

### EXAMPLE 3

### Manufacturing Method 3

Water containing alcohol (water 150 cc, ethanol 150 cc) was used instead of water and agitated by a mixer in the same manner as Example 1. Supernatant was placed in a bottle and left at rest in the sun similarly. The supernatant lessened to 200 cc when 20 days passed and to 120 cc when 28 days passed. The residual liquid smells a high ethanol smell.

### EXAMPLE 4

### Manufacturing Method 4

Ethanol (reagent 1st grade) 180 cc was used instead of water and agitated by a mixer in the same way as Example 1. The supernatant was placed in a bottle and left at rest in the sun similarly, and lessened to 100 cc when 20 days passed and substantially dried when 26 days passed, the residue being sticky and smelling plum liqueur.

### EXAMPLE 5

### Manufacturing Method 5

Live leaves 200 g of Spiraea Thunbergii Sieb. was sufficiently washed with water and placed together with water 1 liter into a pan to be caused to come to a boil and then further continued to be boiled with a lower heat. Heating was stopped when the amount of water become ⅔. The solution obtained from boiling showed a reddish brown color thicker than the color of the liquid when one month passed in Example 1. The solution 50 cc obtained in Example 1 having addition of three drops of herb perfume was agitated. The solution was sprayed over a cloth and the smell of perfume was left on the cloth even when one week passed.

### EXAMPLE 6

### Manufacturing Method 6

In Example 1, live leaves 50 g of Spiraea Thunbergii Sieb. together with live leaves 10 g of Juniperus chinensis L. were processed in the same manner. The resultant solution showed the same insect repelling effect as Example 1.

### EXAMPLE 7

### Confirmation of Safety 1

(Acute oral toxicity examination using male and female mice Limit test—reported by a foundation Japan Food Analysis Center on Dec. 12, 2003) Abstract: Specimen of a capacity 20 ml/Kg (the insect repellent according to the present invention: obtained in Example 1) was applied to the Specimen Dosage group, and water for injection was applied to the Control group in such manner as giving orally to male and female mice in each single time. Observation was made for 14 days, resulting in that there was not seen any cases of abnormality and death in the term of observation (Nov. 20 to Dec. 12, 2003). Thus, it is inferred that LD50 (50% lethal dose) of the specimen in mice with single oral dosages is 20 ml/Kg or more for both of male and female mice.

Weight of mice measured before and after dosage is as shown in Table 1 (male) and Table 2 (female). For both male and female, there was not seen an apparent difference in comparison with the Control group. The Analysis Center also examined existence and non-existence of formaldehyde (Acetylacetone method for absorbance determination) but formaldehyde was not detected (detection limit: 5ppm).

TABLE 1
Change of Weight (Male)

| Groups | before dosage | 7 days after | 14 days after |
|---|---|---|---|
| Specimen | 34.6 ± 0.8(5) | 37.9 ± 1.3(5) | 39.7 ± 1.8(5) |
| Control | 34.8 ± 0.8(5) | 38.7 ± 1.3(5) | 40.7 ± 1.6(5) |

TABLE 2
Change of Weight (Female)

| Groups | before dosage | 7 days after | 14 days after |
|---|---|---|---|
| Specimen | 28.3 ± 0.9(5) | 31.0 ± 3.0(5) | 32.0 ± 3.7(5) |
| Control | 28.3 ± 1.0(5) | 31.9 ± 1.4(5) | 34.7 ± 2.1(5) |

### EXAMPLE 8

### Confirmation of Safety 2

A small amount of the solution obtained in Example 1 (that after one month) was placed in a bottle of a wide mouth, in which two snails were put and observed of their actions. The snails still lived after more than one month in which time no feeding was made. Also, no abnormality was seen on human skin when several days passed after the solution was applied to human body. As foregoing, since ants do not die and not escape even when the solution was sprayed over the marching ants, it is apparent that the solution does not have an acute toxicity to insects.

### EXAMPLE 9

### Confirmation of Effect of the Insect Repellent 1 Mosquitoes

A In a cemetery there were a large number of mosquitoes and people around there cannot bear mosquitoes even in a few minutes in the evening. The solution about 50 cc according to the invention was sprayed over the tombstones, ground and gardening in the cemetery and a neighboring forest in the mountains. During spraying the solution, I was bitten at two points on the body by mosquitoes and evacuated therefrom immediately after spraying the solution. I went the site again 20 minutes after and saw no mosquitoes there. Two hours after also, there were no mosquitoes at the site and the smell of the solution still remained around there. No mosquitoes were seen also in the evening in the next day. Continuation of the insect repelling effect as above is the characteristic of the insect repellent according to the present invention.

The same experiment was performed twice at different cites to find the same result. To be noted is that in the outside in a windy day the solution is to be sprayed from the windward side to have a definite effect.

The solution about 50 cc was sprayed to take care of pine trees in the garden in the similar manner in the daytime. No mosquitoes came near while several dozens of dragonflies came near unexpectedly with the reason being not clarified.
B In the living room of my house, I hitherto used various commercially available items, such as mosquito-incense and the like, against mosquitoes. I hung a cloth, which the solution permeates, at a corner of the room and was not at all bitten by mosquitoes at the night. Two mosquitoes were behind the curtain and flew around but not bit me. The smell of the solution still remained in the room in the next morning. The same effect continued four days while the cloth was slightly dried to which the solution was sprayed three times. Thereafter, no mosquitoes were seen and I found no trace of any dead mosquitoes. I was convinced that no mosquitoes approach the room as far as the smell of the solution even in a trace amount remains in the room. In the summer (2003) I did not at all use any mosquito-incense or other insect repelling agents.
C In one day in the last ten days of July 2003, about 19:30, in a fruit garden near my outside parking lot, I was bitten by mosquitoes three points on the skin per one minute and sprayed the solution twelve times around there and three times on the ground while a mosquito further bit me at one point, and I was quickly evacuated from the place. I went there again 20 minutes after and saw no mosquitoes there.

No mosquitoes came near and I waited for mosquitoes coming for ten minutes with the upper half of my body being naked. There appeared no mosquitoes coming. I laid on a sheet to watch the sky and stars and slept. I woke up at 23:00 to find that my body was not at all bitten by mosquitoes. The solution smelled around there. (no wind in that day)
D In the same room as B, the insect repellent mixing with essential oils of eucalyptus, lemon grass, mint, or clove was sprayed several times. But, the effect was kept only about one hour.

### EXAMPLE 10

### Confirmation of Effect of Insect Repelling Agent 2—Armadillidium

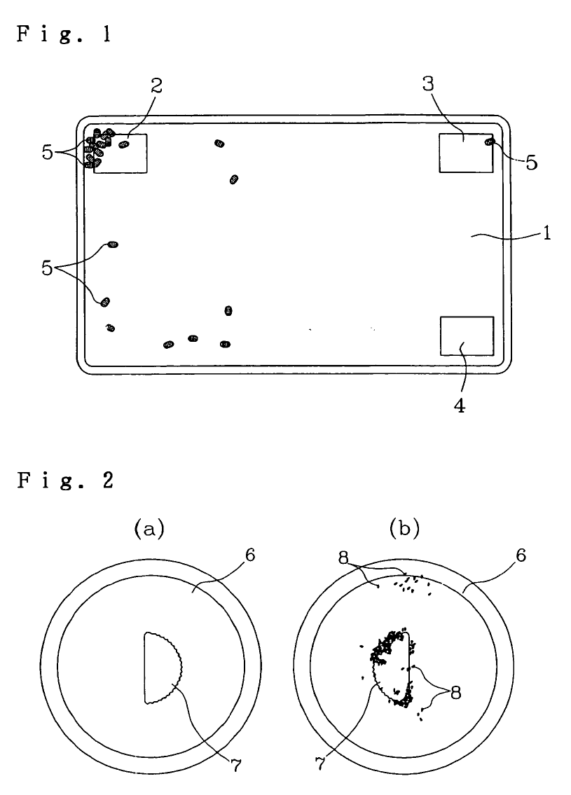

A As seen in FIG. 1, at the three corners of a plastic container 1 (62 cm×40 cm×10 cm depth) were set a cloth 2 (3 cm×2 cm) soaked in water, a cloth 3 sprayed five times with an agent using DEET (10%: spray type), and a cloth 4 soaked in the insect repelling agent obtained in Example 1. 20 of the insects (Armadillidium) were placed in the container 1. The insects began moving all together and acting variously from a corner to another corner or circumferentially. At the time when four hours passed, eight armadillidiums gathered at the cloth permeated with water and stopped there. As time passed, the number of Armadillidium gathering at that corner increased while about 10 armadillidiums always moved around. After eight hours, all except one gathered at the water-permeated cloth.

As a result, it has been found that the insect repellent according to the present invention has a higher insect repelling effect in comparison with the DEET product. Even when the places where the cloths are positioned, directions of the container, and places where the container is positioned are varied, the same result was obtained. After three days passed, the insects stopped moving from the place and most of them went under the cloth. There was no armadillidium dead in the container. Upon ending the experiment, the armadillidiums were released to the outside of house. They all went anywhere.
B Eight armadillidiums were seen under a plant pot and the solution was sprayed there. There was no armadillidiums 10 minutes after. Three armadillidiums and two crickets were under another plant pot and the solution was sprayed similarly. 10 minutes after there appears no armadillidiums but two crickets kept staying. Then, crickets were examined to show no insect repelling effect. The solution was sprayed at a place where the insects always gather. No armadillidiums gathered. Other places had the same results. The smell of the solution remained under the plant pot. In a rainy day, the insect repelling effect reduces since the solution is water-soluble. Armadillidiums do not die even when the solution is directly applied to them.

### EXAMPLE 11

### Confirmation of Effect of Insect Repelling Agent 3—Ants

A As shown in FIG. 2, two small dishes 6 in diameter of about 10 cm on each of which a slice of commercially available boiled fish paste 7 was placed were set on a sink in a kitchen. The two dishes were brought into contact with each other (though separated a little in FIG. 2) and one of the dishes was subjected to spraying of the solution of the insect repelling agent according to the present invention and waited for ants (small house ants, about 0.5 mm in length). On the next day, several hundreds of ants gather on one of the slices of boiled fish paste not subjected to spraying of the solution (FIG. 2(b)) while the other slice of boiled fish paste sprayed with the solution (FIG. 2(a)) had no ants. When two days passed, the status was the same. When the solution was sprayed on the slice of boiled fish paste gathering ants, the ants escaped quickly and all disappeared within 10 minutes. No ants were dead. Furthermore, the solution may be sprayed at a place where ants usually gather in order to prevent ants from coming there.
B When the solution is sprayed over ants in marching, confusion arises at the place and 70% of the ants go backward while the remaining performs marching on a different position. Ants will disappear generally in the meantime. When the solution is preliminarily sprayed over an aimed place, ants do not approach there.

### EXAMPLE 12

### Confirmation of Effect of Insect Repelling Agent 4 A Green Caterpillar

Two green caterpillars on the leaves of cabbage were caught and placed on the ground. The solution was sprayed over a place defining a rectangular shape of 1 cm width and 5 cm length positioned 5 cm forward in front of the escaping caterpillars and extending perpendicularly to the escaping direction of the worms. The green caterpillars highly raised up their heads and turned 90 degrees in direction to avoid the place and escaped. Both of the worms did the same action.

### EXAMPLE 13

### Confirmation of Effect of Insect Repelling Agent 5 Meghimatium (Slugs), Snails, And Butterflies

The similar experiments were performed for slugs (Meghimatium) and snails to have the same result as in Example 12. When the solution was sprayed on the leaves of cabbage, slugs and snails did not come near. Butterflies came flying but not rested on the leaves. Spraying of the solution at the outside of house is possibly washed out with rain, and it is therefore required to select a suitable time or season for the spraying.

### EXAMPLE 14

### Confirmation of Effect of Insect Repelling Agent 6 Spiders

Six nests of spiders were located on a pine tree. The solution was sprayed there and the spiders nests were removed. Thereafter, there were seen no nests of spiders when one week passed.

### EXAMPLE 15

### Confirmation of Effect of Insect Repelling Agent 7 Leaf Beetles

In summer, many leaf beetles come flying to the gate lamp at the entrance of house. The solution was directly sprayed on the gate lamp and the coming flying of leaf beetles notably reduced. Thereafter, the insect repelling effect is long kept but it is more effective to spray the solution once again.

### EXAMPLE 16

### Confirmation of Effect of Insect Repelling Agent 8 Flies

A The solution was sprayed over fresh fish (a horse mackerel, 10 cm length) and the fish was left at rest in the outside of house in the sun. Flies came flying near but not rested on the fish and flew away. Another flies came flying several times similarly and flew away. Thereafter, no flies came flying. Raw waste was positioned outside the house, subjected to spraying of the solution and left at rest. A fly came flying and rested on the raw waste and flew away soon. Another fly came flying and made circuitous flying over the raw waste and flew away. Thereafter, raw waste did not smell a rotting smell and no small flies came flying without generation of maggots. The solution smells around the raw waste. It was confirmed that there is also an effect of preservation from decay and the effect can be kept long (more than five days depending on specific spraying methods).
B No countermeasures were taken against many small flies coming flying to suspended dried persimmons as in other years. A dish on which the solution was applied was placed under the suspended dried persimmons. There after, there appear no small flies.

### EXAMPLE 17

### Confirmation of Effect of Insect Repelling Agent 9 Small Flies

Fruits, apples: When the peel of orange or the like is left at rest to the outside of house, many small flies come flying there after few days. The solution may be sprayed two or three times directly on the peel so that there appear no small flies and no small flies come flying. Since the solution contains a component of keeping humidity, the advantageous effect can be kept for a long time.

1. An insect repellent using as an effective component an extract of leaves, flowers, branches, stems, or roots of the Spiraea group plants, or a treated product of the extract.

2. An insect repellent as set forth in claim 1 wherein the extract is: an extract liquid obtained by soaking leaves, flowers, branches, stems, or roots of the spiraea group plants into water, an organic solvent such as alcohol or the like, or a water-containing organic solvent; an extract liquid obtained by boiling or steaming with these solvents leaves, flowers, branches, stems, or roots of the spiraea group plants; or a supernatant or a squeezed liquid obtained by squeezing leaves, flowers, branches, stems, or roots of the spiraea group plants or grinding together with water and other solvent leaves, flowers, branches, stems, or roots of the spiraea group plants.

3. An insect repellent as set forth in claim 2 wherein the insect repellent comprises a residual substance after squeezing or a residuum obtained in claim 2.

4. An insect repellent as set forth in claim 1, wherein a synthetic or natural insecticide and/or insect repelling agent is added.

5. An insect repellent as set forth in claim 4 wherein the insect repellent is obtained by: mixing a plant having an insect repelling efficiency such as a laurel, Juniperus chinensis L., Pittosporum Tobira Ait., a ginkgo tree, and a mugwort, or a plant having an insecticidal efficiency such as an insect powder plant, NEEM, and Ardisia crispa A. DC., with respect to leaves, flowers, branches, stems, or roots of the spiraea group plants in the proportion of less than 40%; soaking into water, an organic solvent such as alcohol or the like, or a water-containing organic solvent; boiling; squeezing; or grinding.

6. An insect repellent as set forth in claim 4 wherein an extract liquid or a squeezed liquid obtained from leaves or branches of a plant having an insect repelling efficiency such as a laurel, Juniperus chinensis L., Pittosporum Tobira Ait., a ginkgo tree, and a mugwort, or a plant having an insecticidal efficiency such as an insect powder plant, NEEM, and Ardisia crispa A. DC., is mixed with an extract liquid or a squeezed liquid from leaves, flowers, branches, stems, or roots of the spiraea group plants in the proportion of less than 40%.

7. An insect repellent as set forth in claim 1, wherein the insect repellent has addition of a ground material of a small amount of plant including leaves or the like having fragrance such as herbs; is subjected together with such ground material to a grinding process; or has addition of a small amount of an extract, or an essence of a plant having fragrance such as herbs, or a perfume.

8. An insect repellent as set forth in claim 2, wherein a synthetic or natural insecticide and/or insect repelling agent is added.

9. An insect repellent as set forth in claim 3, wherein a synthetic or natural insecticide and/or insect repelling agent is added.

10. An insect repellent as set forth in claim 2, wherein the insect repellent has addition of a ground material of a small amount of plant including leaves or the like having fragrance such as herbs; is subjected together with such ground material to a grinding process; or has addition of a small amount of an extract, or an essence of a plant having fragrance such as herbs, or a perfume.

11. An insect repellent as set forth in claim 3, wherein the insect repellent has addition of a ground material of a small amount of plant including leaves or the like having fragrance such as herbs; is subjected together with such ground material to a grinding process; or has addition of a small amount of an extract, or an essence of a plant having fragrance such as herbs, or a perfume.

12. An insect repellent as set forth in claim 4, wherein the insect repellent has addition of a ground material of a small amount of plant including leaves or the like having fragrance such as herbs; is subjected together with such ground material to a grinding process; or has addition of a small amount of an extract, or an essence of a plant having fragrance such as herbs, or a perfume.

13. An insect repellent as set forth in claim 5, wherein the insect repellent has addition of a ground material of a small amount of plant including leaves or the like having fragrance such as herbs; is subjected together with such ground material to a grinding process; or has addition of a small amount of an extract, or an essence of a plant having fragrance such as herbs, or a perfume.

14. An insect repellent as set forth in claim 6, wherein the insect repellent has addition of a ground material of a small amount of plant including leaves or the like having fragrance such as herbs; is subjected together with such ground material to a grinding process; or has addition of a small amount of an extract, or an essence of a plant having fragrance such as herbs, or a perfume.

15. An insect repellent as set forth in claim 8 wherein the insect repellent is obtained by: mixing a plant having an insect repelling efficiency such as a laurel, Juniperus chinensis L., Pittosporum Tobira Ait., a ginkgo tree, and a mugwort, or a plant having an insecticidal efficiency such as an insect powder plant, NEEM, and Ardisia crispa A. DC., with respect to leaves, flowers, branches, stems, or roots of the spiraea group plants in the proportion of less than 40%; soaking into water, an organic solvent such as alcohol or the like, or a water-containing organic solvent; boiling; squeezing; or grinding.

16. An insect repellent as set forth in claim 9 wherein the insect repellent is obtained by: mixing a plant having an insect repelling efficiency such as a laurel, Juniperus chinensis L., Pittosporum Tobira Ait., a ginkgo tree, and a mugwort, or a plant having an insecticidal efficiency such as an insect powder plant, NEEM, and Ardisia crispa A. DC., with respect to leaves, flowers, branches, stems, or roots of the spiraea group plants in the proportion of less than 40%; soaking into water, an organic solvent such as alcohol or the like, or a water-containing organic solvent; boiling; squeezing; or grinding.

17. An insect repellent as set forth in claim 8 wherein an extract liquid or a squeezed liquid obtained from leaves or branches of a plant having an insect repelling efficiency such as a laurel, Juniperus chinensis L., Pittosporum Tobira Ait., a ginkgo tree, and a mugwort, or a plant having an insecticidal efficiency such as an insect powder plant, NEEM, and Ardisia crispa A. DC., is mixed with an extract liquid or a squeezed liquid from leaves, flowers, branches, stems, or roots of the spiraea group plants in the proportion of less than 40%.

18. An insect repellent as set forth in claim 9 wherein an extract liquid or a squeezed liquid obtained from leaves or branches of a plant having an insect repelling efficiency such as a laurel, Juniperus chinensis L., Pittosporum Tobira Ait., a ginkgo tree, and a mugwort, or a plant having an insecticidal efficiency such as an insect powder plant, NEEM, and Ardisia crispa A. DC., is mixed with an extract liquid or a squeezed liquid from leaves, flowers, branches, stems, or roots of the spiraea group plants in the proportion of less than 40%.
